(19) United States
(12) Patent Application Publication
(10) Pub. No.: US 2017/0238819 A1
(43) Pub. Date: Aug. 24, 2017

(54) SYSTEM AND METHOD TO DETERMINE BLOOD PRESSURE

(71) Applicant: Garmin Switzerland GmbH, Schaffhausen (CH)

(72) Inventors: Alexander J. Waller, Olathe, KS (US); Sean Shin Yuan Yu, Overland Park, KS (US); Jeffrey B. Wigh, Olathe, KS (US)

(21) Appl. No.: 15/435,266

(22) Filed: Feb. 16, 2017

Related U.S. Application Data
(60) Provisional application No. 62/296,966, filed on Feb. 18, 2016.

(51) Int. Cl. A61B 5/021; A61B 5/00; A61B 5/0205

(52) U.S. Cl. CPC A61B 5/02125; A61B 5/0205; A61B 5/4875; A61B 5/742; A61B 5/7246; A61B 5/681; A61B 5/02427

(57) ABSTRACT

A system, method, and device for monitoring a physiological characteristic of a user includes a wearable monitoring device including one or more LEDs configured to emit light toward a user's skin tissue and two or more sensors laterally disposed along a longitudinal axis of an extremity of a user. Each sensor generates a signal based on an intensity of received light from a location against or adjacent to the sensor where a pulse wave resulting from a user's heart beat passes. A processor calculates one or more physiological characteristics of the user, such as blood pressure or stress, based on the generated signal from each sensor and a lateral distance therebetween.

### CROSS-REFERENCE TO RELATED APPLICATIONS

This patent application claims the filing benefit of U.S. Provisional Patent Application entitled, “Using Optical Sensors to Determine Blood Pressure,” Ser. No. 62/296,966, filed Feb. 18, 2016; the contents of which are expressly incorporated by reference herein.

### BACKGROUND

Many systems for monitoring cardiac characteristics of a user implement various combinations of mechanical, electrical, and optical components. For example, some blood-pressure monitors utilize an inflatable cuff that contracts an opening around a user's limb or portion thereof. Attachment of the cuff may be cumbersome and impracticable for use over periods of prolonged duration or high levels of physical activity. Further, inflation of the cuff typically creates a noticeable tension around the user's limb and prolonged use of the cuff is not intended or anticipated during the day or especially during sleep.

Unlike a conventional cuff or a cardiac monitoring system in a patient-care, medical environment in which a user may be sedentary or otherwise at rest (i.e., a patient receiving medical care while seated or lying in bed), a user may desire cardiac information while engaged in most daily activities (e.g., typing, driving, writing, walking, running, riding, swimming, rowing, etc.). Utilization of conventional monitoring systems for determining cardiac and physiological characteristics (heart-rate, blood pressure, hydration status) in a medical environment are ineffective and/or impracticable for use during most daily physical activities or while sleeping.

### SUMMARY

In one aspect of the invention, a wearable monitoring device capable of being attached to an extremity of a user and determining a physiological characteristic of the user, includes a housing containing a first photodiode and a second photodiode, at least one LED, and a processor. The at least one LED is configured to output light into the user's extremity, wherein the first photodiode is positioned proximate to the at least one LED and configured to generate a first PPG signal based on a reflection of light from a detected pulse wave, and wherein the second photodiode is positioned proximate to the at least one LED and spaced a lateral distance from the first photodiode within the housing and configured to generate a second PPG signal based on the reflection of light from the detected pulse wave. The processor is coupled to the first and second photodiodes and configured to receive the first and second PPG signals, determine a pulse-transit-time of the user based on the generated first and second PPG signals, and calculate the physiological characteristic of the user based on the calculated pulse-transit-time and the lateral distance between the first and second photodiodes, and wherein the second photodiode detects the pulse wave after the first photodiode when the device is attached to the extremity of the user.

In another aspect of the invention, a wearable monitoring device capable of being attached to the extremity of a user and determining a physiological characteristic of the user, includes a first photodiode spaced apart a lateral distance from a second photodiode when the device is attached to the extremity of the user. The lateral distance extends along the longitudinal axis of the extremity of the user between the first photodiode and the second photodiode. The device further includes at least one LED proximate the first photodiode and the second photodiode and configured to output light to be received by the first and second photodiodes. The first photodiode detects a pulse wave of the user and generates a first PPG signal, and the second photodiode detects the pulse wave of the user and generates a second PPG signal. A processor coupled to the first and second photodiodes and configured to calculate the physiological characteristic of the user based on the generated first and second PPG signals and the lateral distance between the first and second photodiodes.

In a further aspect of the invention, a wearable monitoring device capable of being attached to the extremity of a user and determining blood pressure of the user, includes a pair of photodiodes spaced apart a lateral distance, such that when the device is attached to the extremity of the user, the lateral distance extends along the longitudinal axis of the extremity of the user between a first photodiode and a second photodiode. The device further includes at least one LED proximate the first photodiode and the second photodiode and configured to output light received by the first and second photodiodes. The first photodiode detects a pulse wave of the user and generates a first PPG signal, and the second photodiode detects the pulse wave of the user and generates a second PPG signal. The device further includes a processor coupled to the first and second photodiodes. Instructions are stored on a memory coupled to the processor, wherein the instructions when executed by the processor, cause the processor to receive the first PPG signal, receive the second PPG signal, calculate a pulse-transit-time (PTT) of the pulse waved based on the first and second PPG signals, calculate a pulse velocity of the pulse wave (PWV) based on the calculated pulse-transit-time and the lateral distance between the first and second photodiodes, calculate the blood pressure of the user based on the calculated pulse velocity, and display the calculated blood pressure.

### BRIEF DESCRIPTION OF THE DRAWINGS

FIGS. 1A and 1B depict configurations of optical sensor placement of known exemplary wearable monitoring systems capable of being attached proximate an extremity of a user;

FIG. 2 depicts a skeletal portion of an exemplary extremity of a user where an embodiment of the present invention can be attached;

FIG. 3 depicts an exemplary process for monitoring a physiological characteristic of a user as described herein;

FIGS. 4A, 4B, and 4C depict various configurations for placement of the optical sensors of the wearable monitoring system of the present invention as described herein;

FIG. 5 depicts an exemplary placement of optical sensors and LEDs of an embodiment of the wearable monitoring system of the present invention as described herein;

FIG. 6A depicts two PPG signals generated by two optical sensors positioned a lateral distance apart along a longitudinal axis of the user's wrist, according to embodiments of the present invention as described herein;

FIG. 6B depicts a differential signal resulting from the two PPG signal depicted in FIG. 6A;

FIG. 7 is an illustration depicting one embodiment of a system capable of executing the process for monitoring a physiological characteristic of a user as described herein;

FIG. 9 depicts differential and filtered signals associated with a user;

FIGS. 8A and 8B depict views of one embodiment of the mobile monitoring system of the present invention as described herein;

FIGS. 10A, 10B, 10C, and 10D depict various information displayed on one embodiment of the mobile monitoring system of the present invention as described herein; and,

FIGS. 11A, 11B, and 11C depict various information displayed on another embodiment of the mobile monitoring system of the present invention as described herein.

### DETAILED DESCRIPTION

Embodiments of the present invention provide an improved cardiac monitoring system with the features described herein. The improved cardiac monitoring system may include a wearable monitoring device having two or more optical sensors (e.g., photodiodes, photosensors, etc.) that are positioned in line with an extremity of a user, such as the user's wrist, when the wearable device is attached to the user's extremity. For example, a housing of the wearable monitoring device may include on a rear surface of the housing, which contacts the user's wrist when the wearable monitoring device is work on the user's wrist, two optical sensors spaced apart a lateral distance along or aside a longitudinal axis of the user's wrist such that one of the two optical sensors is closer to the user's elbow than the other optical sensor. The optical sensors may be positioned along the user's wrist or forearm such that a pulse wave (blood cells pushed as the heart contracts and expands) passes one optical sensor before the pulse wave passes the other optical sensor with each contraction and expansion of the user's heart.

Unlike certain conventional wearable monitoring devices having two optical sensors oriented perpendicular to the direction of blood flow on a rear surface of a housing (i.e., both optical sensors are an equal distance from the user's elbow) such that the user's pulse wave pass each optical sensor at the same time, embodiments of the present invention enable a processor of the wearable monitoring device to identify a pulse wave at two locations corresponding to the first and second optical sensors. In embodiments, the first optical sensor is positioned at a first location closer to the user's elbow than the second optical sensor positioned at a second location, which is closer to the user's fingertips and hand. As a result, a pulse wave originating from the user's heart and resulting from an expansion and a contraction of the heart will arrive at the first location associated with the first optical sensor before the pulse wave arrives at the second location associated with the second optical sensor.

The lateral distance between the first and second locations is known based on a lateral distance between the first optical sensor and the second optical sensor. A processor may determine a length of time that passed between the pulse wave arriving at the first location and the second location (pulse-transit-time) as well as a velocity of the pulse wave based on the time over which the pulse wave traveled the lateral distance between the first and second optical sensors (pulse wave velocity). In embodiments, the processor may determine, or utilize data stored in memory to determine, a physiological characteristic (e.g., blood pressure, stress, etc.) of the user based on the pulse-transit-time or the pulse wave velocity. For instance, the processor may utilize data stored in memory including a correlation between systolic pressure, diastolic pressure, and/or mean arterial pressure with pulse-transit-time or pulse wave velocity.

Some conventional cardiac monitoring systems determine a user's heart-rate by utilizing electrodes (e.g., conductive pads) to sense electrical signals in a user's body, such as the chest area, and generate an electrocardiogram (ECG) signal. A processor determines the user's heart-rate based on the ECG signal. Other heart-rate monitoring systems utilize light of certain wavelengths and optical sensors (e.g., photosensors, photodiodes, etc.) that generate a photoplethysmogragh (PPG) signal, wherein a processor determines the user's heart-rate based on the generated PPG signal.

Additionally, some conventional cardiac systems utilize biometric telemetry and utilize a combination of electrodes positioned near a user's chest a fingertip pulse oximeter including an optical sensor and light-emitting diodes (LEDs) to determine a user's heart-rate and blood pressure. Specifically, the electrodes positioned near a user's chest generate an ECG signal and the fingertip pulse oximeter generates a PPG signal by outputting light in the red, blue, and/or infrared spectrum into the user's fingertip and receiving reflections of the outputted light. A processor determines the user's heart-rate and blood pressure based on an estimated distance between the user's heart and fingertip(s). Estimates of the distance between the user's heart and fingertips are commonly based on the user's height. The processor may utilize an ECG signal generated by an electrode positioned near a user's chest to detect an electrical signal associated with a contraction of the user's heart, which may precede the actual contraction of the heart. This time delay, e.g., “pre-ejection period,” between the excitation of the user's heart-beat and the contraction of the heart may vary among individuals and may be affected by medical treatments, e.g., prescribed drugs. The combined usage of electrical and optical equipment in such conventional systems complicates the process of determining cardiac characteristics of a user based on a process requiring measurement and analysis, e.g., comparison, of respective electrical and optical signals generated by components utilizing different technological means. Additionally, due to the type of equipment and environment necessary to generate the ECG and PPG signals and the placement thereof (e.g., chest, fingertip), these conventional systems are typically utilized in patient-care environments (e.g., a hospital, clinic, etc.) and are generally considered impracticable for use during in environments associated with active or mobile users.

Other optical heart-rate monitoring systems are embedded within a wearable device, such as a wrist-watch (e.g., Garmin Forerunner®) or bracelet (e.g., Garmin Vivoactive®). These systems are often referred to as wrist-based heart-rate monitors (WHRMs). Known WHRM systems typically include a single optical sensor that is positioned on a rear surface of a wearable monitoring device housing at a location corresponding to the user's main arteries and veins. Known WHRM systems that include a plurality of optical sensors position the optical sensors perpendicular to the direction of blood flow such that the user's blood cells pass each sensor at the same time (i.e., the optical sensors are an equal distance from the user's elbow). The WHRM systems typically employ one or more LEDs that are directed to emit light toward a user's wrist and one or more optical sensors are positioned to measure reflections of the light from the user's skin that was emitted from the LEDs. The one or more optical sensors generate a PPG signal based on the reflected light received by the optical sensor(s). A processor of the WHRM or a related system can analyze the PPG signal over a period of time to calculate the user's heart-rate and/or other physiological characteristics.

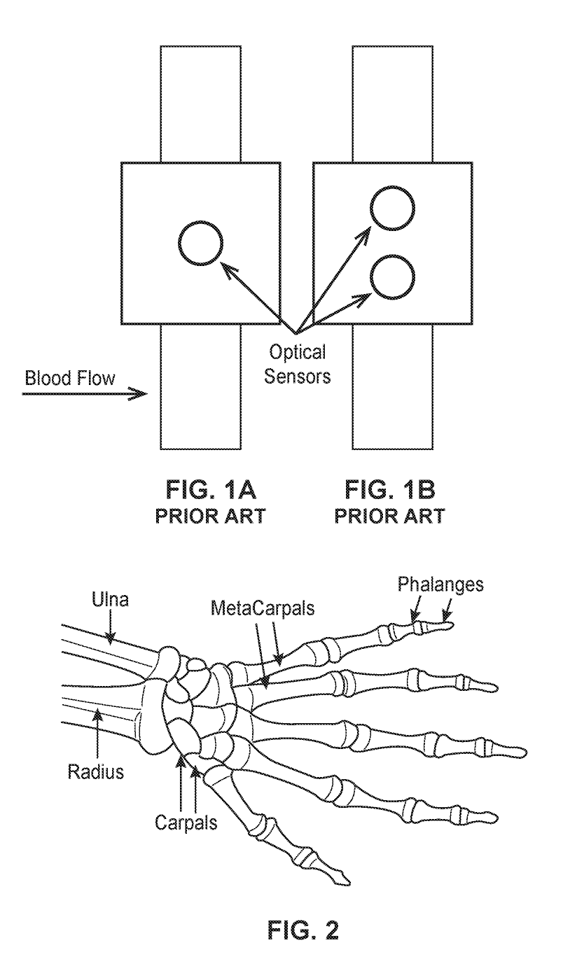

Examples of known layouts of optical sensors (e.g., photodiodes, photosensors, etc.) are shown in FIGS. 1A and 1B without depicting LEDs that emit light received by the optical sensors, where one or more optical sensors are positioned about the user's wrist (see FIG. 2, depicting a partial skeletal diagram of an extremity, e.g., limb, arm, wrist, hand). One or more LEDs may be positioned at any location proximate to the optical sensors to output sufficient light to enable receipt of light reflections from the user's wrist. In FIG. 1A, a single optical sensor may be positioned between a user's ulna and radius bones, and in FIG. 1B, two optical sensors may be positioned in line perpendicular to the extremity. In the dual optical sensor configuration of FIG. 1B, one optical sensor is positioned closer to the user's ulna bone and a second optical sensor is positioned closer to the user's radius bone. Such positioning of the two optical sensors may enable detection of a more prominent or stronger arterial pulse within the limb. For example, a processor of the wearable monitoring device may select a higher-quality PPG signal generated by one of the two sensors (e.g., having a higher amplitude, expected data pattern, etc.). As shown in FIG. 1B, each of the two optical sensors are positioned at substantially the same lateral distance between the user's elbow and fingertips (phalanges). That is, the two optical sensors are aligned perpendicular to a longitudinal axis, e.g., general lateral path of blood-flow, within the extremity, wherein neither optical sensor is closer or nearer to the elbow or fingertips than the other optical sensor.

When a user's heart beats (by expanding to draw in blood cells and contracting to push out blood cells), a pulse wave of blood cells travels from the heart through the user's arteries and blood vessels. The processor disclosed herein is configured to determine a physiological characteristic of the user (e.g., blood pressure (systolic, diastolic, and by extension the mean arterial pressure (MAP)), stress, etc.). In embodiments, the processor may determine the physiological characteristic based on a correlation stored in memory between a velocity of a pulse wave, biometric or physical information (e.g., age, height, weight, gender, body mass index (BMI), bone density, etc.), activity type (e.g., running, walking, sitting, etc.) and an estimated blood pressure for a user.

As disclosed herein, two or more optical sensors may be separated by a lateral distance, which may be limited by a width or diameter of the wearable monitoring device housing, along an extremity (e.g., wrist) of a user to identify the presence of the pulse wave caused by the user's wear beat as it passes by each of optical sensors. The processor may utilize or retrieve from memory a known distance between the two or more optical sensors and a PPG signal generated by each optical sensor to determine a time at which the pulse wave arrived at and passed by each optical sensor. The processor may be further configured to calculate a pulse-transit-time (PTT) and/or a pulse-wave-velocity (PWV) of the pulse wave based on the PPG signals and the lateral distance between two or more optical sensors.

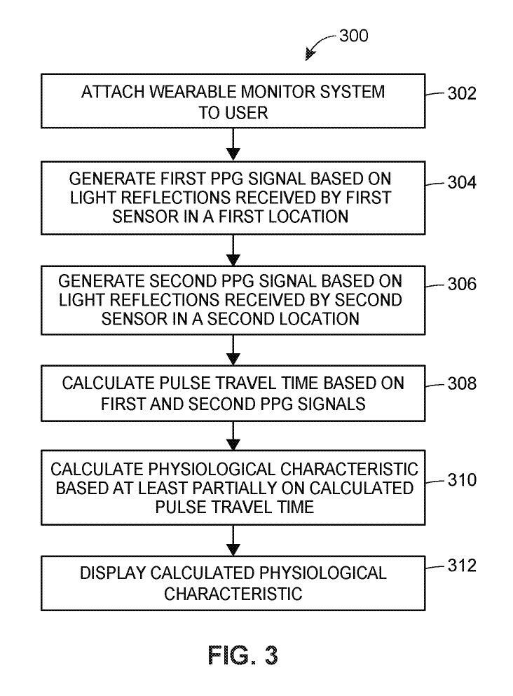

FIG. 3 illustrates a method flow 300 for a monitoring a physiological characteristic (e.g., blood pressure, stress, etc.) of the user, according to embodiments of the present invention. In embodiments, method 300 may begin when a wearable monitoring device is attached to an extremity of a user, such as the user's wrist (block 302). A housing of the wearable monitoring device includes two or more optical sensors (photosensors, photodiodes), which when attached to the user's extremity, are positioned on a rear surface of the housing in line with the extremity. That is, for a wrist-worn wearable monitoring device, the optical sensors are spaced apart a lateral distance along or aside a longitudinal axis of the wrist, which extends along the generally lateral path of blood flow from the shoulder at one end towards the wrist, hand and fingers on the other end. The extremity may generally extend from the shoulder at one end, through the upper and lower arms, and toward the wrist, hand, and fingers at the other end (e.g., along the forearm or wrist). For example, two optical sensors may both be located near an end of the extremity, such as the wrist, or the two optical sensors may be located several inches apart. The two optical sensors may be located several inches apart when one optical sensor is located between the user's shoulder and elbow (e.g., upper arm) and another optical sensor may be located between the user's elbow and wrist or fingers (e.g., lower arm or forearm).

Each optical sensor receives reflections of light emitted (output) by one or more light-emitting diodes (LEDs) positioned proximate to the optical sensor from the skin tissue proximate thereto. Each optical sensor generates a photoplethysmogragh (PPG) signal based on the intensity of light received by the optical sensor. A first optical sensor will generate a first PPG signal based on light reflections received by the first optical sensor from a first location proximate to the first optical sensor. (block 304). A second optical sensor will generate a second PPG signal based on light reflections received by the second optical sensor from a second location proximate to the second optical sensor. (block 306). The PPG signal generated by each optical sensor will change when the pulse wave arrives at and passes through a location proximate to the optical sensor because light reflected from the user's skin may increase when a detected pulse wave (e.g., blood cells) travelling from the heart to the end of the extremity arrives at and passes through the location. The PPG signal may be generated by each optical sensor continuously or periodically. A processor of the wearable monitoring device may receive and analyze each PPG signal to identify presence of the pulse wave at the location corresponding to the position of the optical sensor that generated the PPG signal.

In embodiments, the processor may utilize or retrieve from memory a known lateral distance between the two optical sensors on the rear surface of the wearable monitoring device housing. The lateral distance between two optical sensors may be measured from the center of each optical sensor or from a point along the perimeter of each optical sensor closest to the other optical sensor. The lateral distance may be stored in a memory enclosed within the wearable monitoring device and used by the processor to calculate a pulse-transit-time (PTT) and a pulse-wave-velocity (PWV) of a pulse wave.

In configurations where the wearable monitoring device includes a housing, for example, a watch, having a width or diameter to include two or more optical sensors on the rear surface of the housing, the lateral distance between two optical sensors may be any distance limited by the width or diameter (lateral dimension) of the watch housing. For instance, the lateral distance between two optical sensors may be 5-20 mm for a watch housing having a width or diameter of 25 mm. Similarly, the lateral distance between two optical sensors may be 5-42 mm for a watch housing having a width or diameter of 38-51 mm. In another configuration, the two optical sensors may be included within a strap used to attach the mobile monitoring system, e.g., watch, to the extremity of the user.

Other configurations may include each optical sensor embodied within a separate housing and/or strap, wherein the lateral distance between the two optical sensors may be 2-24 inches and the optical sensors are communicatively coupled to a processor that may receive a PPG signal from each optical sensor and a lateral distance between the optical sensors. For example, in embodiments, a first optical sensor may be located between the user's shoulder and elbow (e.g., upper arm) and a second optical sensor may be located between the user's elbow and wrist or fingers (e.g., lower arm or forearm). The first optical sensor may be positioned on an inner (lower) surface of a wearable monitoring device with a housing in the form of a band (with or without a display device) such that the first optical sensor is positioned against or adjacent to the user's skin at a location between the user's shoulder and elbow and configured to generate a first PPG signal based on an intensity of light reflections received from the user's skin at that location. The second optical sensor may be positioned on an inner (lower) surface of a wearable monitoring device with a housing in the form of a watch device (with a display device) or band (with or without a display device) such that the second optical sensor is positioned against or adjacent to the user's skin at a location between the user's elbow and wrist or fingers and configured to generate a second PPG signal based on an intensity of light reflections received from the user's skin at that location. A processor of the monitoring system may be communicatively coupled with the first optical sensor and the second optical sensor and configured to receive the first and second PPG signals, respectively, and utilize the received PPG signals to determine a physiological characteristic of the user.

In embodiments, the processor may utilize or retrieve from memory a known distance between the two or more optical sensors and a PPG signal generated by each optical sensor to determine a time at which the pulse wave arrived at and passed by each optical sensor, a pulse-transit-time (PTT) based on the PPG signals and the lateral distance between two or more optical sensors. (block 308). The PTT is the length of time that passed for the user's pulse wave (e.g., blood cells) to travel the lateral distance between the two optical sensors. The two or more optical sensors may be synchronized in time to enable the processor to compare the PPG signals generated and provided by each optical sensor to the processor for the calculation of the PTT and/or PWV of the pulse wave.

For some users, the PTT may be 50-100 ms and the PTT may be affected by physiological characteristics of the wearer. For example, blood cells of the pulse wave may pass the lateral distance from the first optical sensor to the second optical sensor in a shorter duration of time when the user's blood pressure is elevated as compared to a longer PTT for blood cells of the pulse wave to travel the lateral distance between the two optical sensors when the user's blood pressure is comparatively lower. Calculated or estimated PTTs may be stored on a memory device of the wearable monitoring device and the processor may reference the stored PTT information to physiological characteristic data stored therewith (block 310). The processor may be configured to utilize the PPG signals received from each optical sensor to determine the physiological characteristic of the user, such as blood pressure (systolic, diastolic, and mean arterial pressure (MAP)), or a stress calculation.

The physiological characteristic may include blood pressure, heart-rate, hydration status, stress, wellness, etc. The processor may determine the physiological characteristic based on a correlation stored in memory between a PTT and/or PWV of a pulse wave, biometric or physical information (e.g., age, height, weight, gender, body mass index (BMI), etc.), and a physiological characteristic, such as an estimated blood pressure, heart-rate, stress, or stress for the user. In embodiments, the processor may also consider other factors, such as the current activity type (e.g., running, walking, sitting, etc.) of the user to determine the physiological characteristic. The processor may utilize information stored in a memory device relating to biometric or physical information corresponding to a group of individuals with similar or different biological and/or physiological characteristics as the user of the wearable monitoring device and/or a group of individuals engaged in similar activities as the current activity of the user.

The processor may calculate the PWV by dividing the known lateral distance between the two optical sensors by the PTT, which is a length of time determined by the processor as having passed for the pulse wave to travel from the first optical sensor to the second optical sensor. PTT and PWV may be included in the stored correlated physiological characteristic data, which may be, for example, a chart or table associating a physiological characteristic (e.g., blood pressure values (e.g., systolic and/or diastolic), stress, etc.) to a determined heart-rate, hydration status, wellness, etc., as well as other biometric or physical information factors, such as age, height, weight, gender, body mass index (BMI), bone density, current activity type (e.g., running, walking, sitting, etc.), with PTTs and/or PWVs. For instance, the processor may utilize the stored correlated physiological characteristic data to determine that a user's blood pressure increases as the PTT decreases (and PWV increases) and that the user's blood pressure decreases as the PTT increases (PWV decreases). The processor may utilize the stored correlated physiological characteristic data to determine a blood pressure for a user based on the calculated PTT and/or PWV determined by the processor based on the PPG signals provided by the two optical sensors and the lateral distance between the optical sensors.

In embodiments, the wearable monitoring device may include a display device having a user interface. The processor may present on the user interface measured and/or determined physiological characteristics (e.g., blood pressure, stress, etc.), PTT, PWV, PPG, oxygen saturation. (block 312). In embodiments, the measured and/or determined physiological characteristics may be transmitted via wired and/or wireless communication to a remote display device or user interface (e.g., a smartphone display) for viewing by a user.

The monitoring system may determine one or more physiological characteristics of the user based on various combinations of PPG and ECG signals. For instance, the processor of the monitoring system may utilize two PPG signals, as detailed above. Alternatively, the processor may utilize two ECG signals and/or a PPG signal and an ECG signal to measure and/or determine one or more physiological characteristics (e.g., blood pressure, stress, etc.), PTT, PWV, PPG, oxygen saturation. The one or more LEDs and the two or more optical sensors may be positioned at different locations along a user's extremity, e.g., limb, to generate the PPG signals provided by each optical sensor for determining the PTT. In embodiments, the different locations one or more LEDs and the two or more optical sensors may be on the rear surface of the wearable monitoring device housing at which the components are positioned against the wrist of a user. Alternatively, the processor may receive a PPG signal from an optical sensor positioned on a user's wrist and an electrocardiogram (ECG) signal from electrodes positioned elsewhere on a user's body, such as the user's chest or upper arm. The processor may estimate a distance between the user's wrist and the location of the electrodes and the PPG and ECG signals may be utilized to determine a PTT and/or PWV for a pulse wave resulting from the user's heart-beat. The processor may then determine one or more physiological characteristics based on the PTT and/or PWV. For instance, the processor may utilize correlation data stored in memory to determine a physiological characteristic, such as blood pressure, of the user.

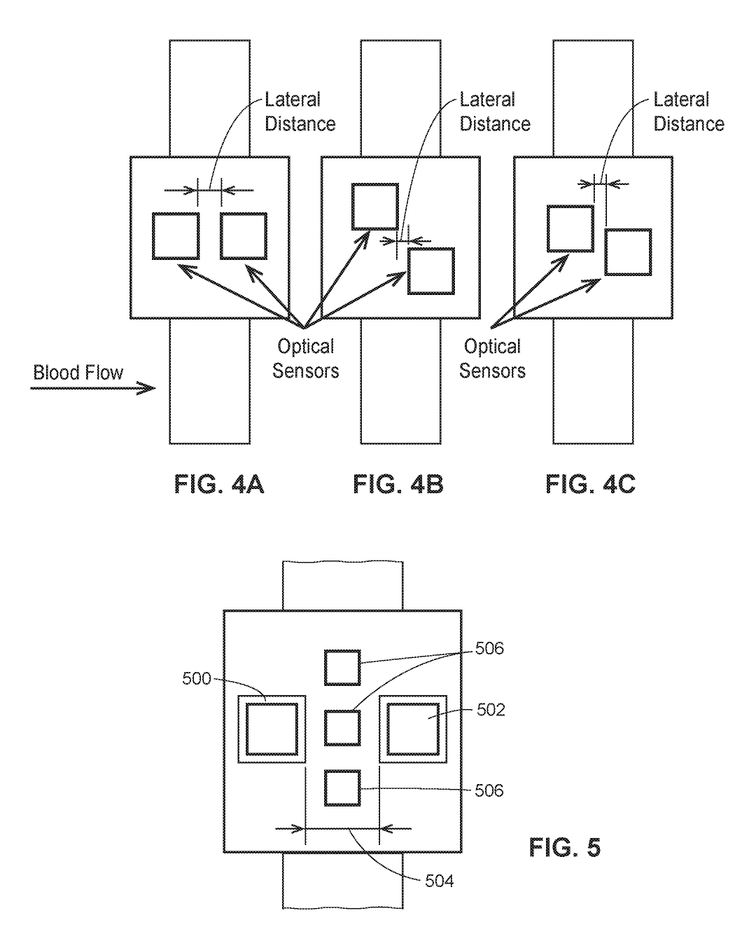

FIGS. 4A, 4B, and 4C depict different configurations of two optical sensors for positioning on a rear surface of a wearable monitoring device housing against a user's wrist or any other portion of the user's extremity or limb (e.g., forearm, ankle, etc.). In accordance with embodiments of the present invention, two or more optical sensors are positioned on the user's skin tissue along an arterial path that is substantially parallel with a longitudinal axis of the user's wrist or other extremity. The two optical sensors are horizontally aligned within a width of the rear surface of the wearable monitoring device housing and separated by a lateral distance that is substantially parallel with the longitudinal axis of the wrist or other extremity when the wearable monitoring device is worn by a user. Each optical sensor independently samples skin tissue adjacent to the optical sensor to detect a pulse wave travelling from the heart to the location of the optical sensor.

Although the two optical sensors are horizontally positioned in FIGS. 4A, 4B, and 4C, the two optical sensors may be vertically offset with respect to each other and locations that may not adversely affect detection of the pulse wave as it travels by the optical sensors positioned along the limb and the subsequent calculation by the processor of the one or more physiological characteristics. That is, one optical sensor may be positioned closer to the ulna bone and the other optical sensor may be positioned closer to the radius bone as shown in FIGS. 4B and 4C, and visa-versa. As discussed herein, the processor of the monitoring system is configured to utilize the known lateral distance (horizontal separation) between the two optical sensors to determine a PTT and/or PWV of a pulse wave, and subsequently, a physiological characteristic, such as blood pressure, for a user by performing the techniques disclosed herein.

In embodiments, the monitoring system may include a wearable monitoring device including at least one LED positioned sufficiently near two optical sensors to enable the optical sensors to operatively sense reflections of light initially emitted from the at least one LED and reflected back from the user's skin tissue. In some embodiments, a plurality of LEDs may be positioned around each and/or both optical sensors such that the optical sensors sense reflected light emitted from the plurality of shared LEDs. For example, in FIG. 5, the wearable monitoring device housing may include two optical sensors 500, 502 aligned horizontally and a plurality of LEDs 506 aligned vertically and positioned an equal distance between the two optical sensors 500, 502. The plurality of LEDs 506 may extend between the user's ulna and radius bones such that the light sensed by each optical sensor 500, 502 is provided by the shared LEDs 506. In another embodiment, the wearable monitoring device may include a combination of two or more optical sensors and one or more unshared LEDs that produce light that may be concentrated at a side of the housing corresponding to each optical sensor such that one optical sensor does not receive reflections from the unshared LED emitting light that may be reflected to another optical sensor.

The processor performs operations on and analyzes the PPG signals generated and provided by the two optical sensors. For example, the processor may receive a first PPG signal from the first optical sensor (e.g., positioned closer to a user's elbow than a second optical sensor), and a second PPG signal from the second optical sensor (e.g., positioned closer to the user's finger tips than the first optical sensor). Because the pulse wave of blood cells that is identified and utilized by the processor to determine physiological characteristics generally flows away from the heart and towards the fingertips with each heart-beat, the unique moment (or sequence of PPG signal values(s)) of a pulse wave of blood cells arrives at and passes by the first optical sensor that is relatively closer to the user's elbow before the pulse wave of blood cells will arrive at and pass by the second optical sensor that is located relatively closer to the user's fingertips. The processor may compare the PPG signals to determine the PTT (e.g., time delay) between a time when a unique moment (or sequence of PPG value(s)) identified in the first PPG signal is identified and a time when the unique moment is subsequently identified in the second PPG signal. For example, the processor may determine a mathematical difference (e.g., subtraction) between the two times at which the pulse wave passed by the first optical sensor and the second optical sensor, respectively.

In embodiments, the processor may control an optical sensor to utilize a higher sampling rate, such as when the two or more optical sensors are positioned closer to each other, to enable the processor to more clearly identify in a PPG signal generated by the controlled optical sensor a light intensity associated with a pulse wave passing by the optical sensor. The use of a higher sampling rate by an optical sensor may enable the processor to differentiate a peak of a pulse wave when the pulse wave was located at the location of the first optical sensor from the peak of the pulse wave when the pulse wave was located at the location of the second optical sensor, which will occur shortly after the pulse wave passes by the first optical sensor.

In embodiments, the lateral distance between two optical sensors may be reduced such that a pulse wave may arrive at a location of a first optical sensor and a second optical sensor may begin to detect (sense) the presence of the pulse wave before the pulse wave has completely passed by the first optical sensor. Use of a high sampling rate for the optical sensors also enables the processor to sample a higher resolution of the PPG signal received from an optical sensor. The processor may identify and determine the PPG signal peaks with better precision, which enables the peak detection and cross-correlation algorithms implemented by the processor to be more accurate. Each optical sensor may generate a PPG signal by sampling light reflected from a user's skin at a high sampling frequency, e.g., (50-2,000 Hz) and provide the PPG signal to the processor and/or a memory device of the monitoring system. The memory device is communicatively coupled with the processor and may be included within the wearable monitoring device housing and/or may be remote to the wearable monitoring device.

In embodiments, once the processor receives the PPG signals from each optical sensor, the processor may compare each PPG signal and determines how much of a temporal shift is necessary to align the PPG signal received from the second optical sensor (further from the heart than the first optical sensor) with the PPG signal received from the first optical sensor (closer to the heart than the second optical sensor). The determination of this temporal shift may provide the processor with the PTT associated with a pulse wave resulting from a heart-beat of a user. Because a known lateral distance between the two optical sensors may be utilized or retrieved from memory by the process, the processor can subsequently determine the PWV associated with a pulse wave by dividing the lateral distance between the two optical sensors by the PTT.

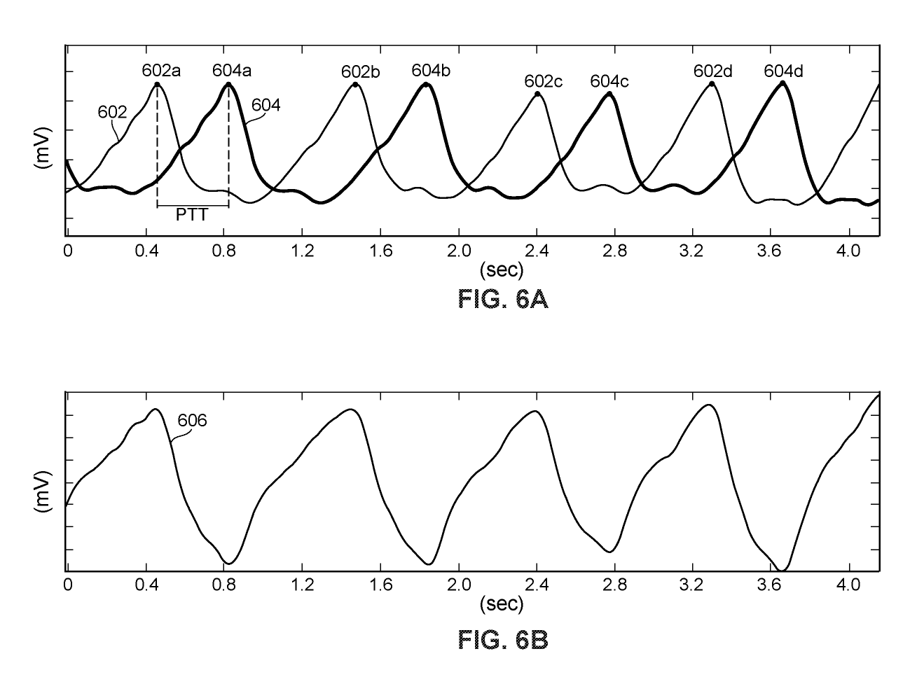

As shown in FIG. 6, the processor of the wearable monitoring device may receive a PPG signal from each of the two optical sensors positioned against or adjacent to the user's skin and positioned a lateral distance apart on a rear surface of a housing of the wearable monitoring device. A first optical sensor generates and outputs a first PPG signal 602. A second optical sensor generates and outputs a second PPG signal 604. The processor may analyze the first PPG signal 602 to identify peaks 602a, 602b, 602c, and 602d of the first PPG signal 602. Similarly, the processor may analyze the second PPG signal 604 to identify peaks 604a, 604b, 604c, and 604d of the second PPG signal 604.

In embodiments, the processor may determine a PTT of one or more pulse waves by calculating a length of time that passed between peaks 602a and 604a for a first pulse wave, peaks 602b and 604b for a second pulse wave, peaks 602c and 604c for a third pulse wave, and peaks 602d and 604d for a fourth pulse wave, respectively. In embodiments, the processor may implement a technique of moving (or sliding) second PPG signal 604, while holding first PPG signal 602 in place, until the two PPG signals 602, 604 substantially overlap to determine a PTT for a pulse wave. The processor may then determine the amount of movement (or sliding) of second PPG signal 604 was required for the second PPG signal 604 to substantially overlap first PPG signal 602 and identify a PTT based on the determined amount of movement (or sliding).

In embodiments, the processor may determine a PTT for each of a plurality of pulse waves and determine a PTT for use with determining a physiological characteristic, such as blood pressure or stress. For example, the processor may determine a PTT for each of the four pulse waves depicted in FIG. 6A (the amplitude of the PPG signal may be expressed in millivolts (mV)) and calculate an average PTT using all four PTT calculations for use with determining a physiological characteristic, such as blood pressure. Alternatively, the processor may utilize a standard deviation of all four PTT calculations to determine a PTT value for use with determining a physiological characteristic, such as blood pressure.

The processor may generate a differential signal 606, as depicted in FIG. 6B, based on first PPG signal 602 and second PPG signal 604. The differential signal 606 may be utilized in embodiments utilizing a single PPG signal for determining a heart-rate or other physiological information about the user wearing the wearable monitoring device.

In embodiments, the processor may utilize information stored memory to determine a physiological characteristic, such as blood pressure, or stress. For example, the processor may determine an estimated current blood pressure for a user based on the determined PWV and a curve-fit of physiological characteristic data stored in memory that may account for variations amongst users relating to biometric or physical information (e.g., age, height, weight, gender, body mass index (BMI), bone density, activity type (e.g., running, walking, sitting, etc.), and/or general health of a user. In embodiments, the processor may retrieve from the memory device blood pressure data and information collected using a clinical blood pressure upper arm cuff and periodically calibrate the stored curve-fit of physiological characteristic data based on the blood pressure data and information collected using the arm cuff.

An accelerometer may also be incorporated within wearable monitoring system during monitoring of the physiological characteristics, e.g., blood pressure measurements, for consideration of whether the user is at rest or engaged in a physical activity. Through the use of data provided by the accelerometer, the processor may identify the type of physical activity of the user is engaged in and thereby adjust the sampling rate (cycle or interval of blood pressure samples) utilized by the two or more optical sensors and the processor accordingly. The processor may also perform blood pressure measurements at pre-scheduled increments of time during a specified activity. For instance, if the user is riding a bike, the processor may be configured to perform a blood pressure measurement every 30 seconds during the ride. Alternatively, if the user is sedentary, the processor may be configured to perform a blood pressure measurement every 10 minutes.

Additionally, the processor may store in a memory device a pre-ejection period, which is a time delay between excitation of the user's heart-beat and contraction of the user's heart, and utilize the pre-ejection period to determine a physiological characteristic (e.g., blood pressure, stress, etc.) for the user. The pre-ejection period may vary among individuals and may be estimated by the processor based on biometric or physical information (e.g., age, height, weight, gender, body mass index (BMI), bone density, etc.). The personal variations may be stored in the memory device as correlated physiological characteristic data and utilized by the processor for consideration in the determination of the user's physiological characteristic, such as blood pressure.

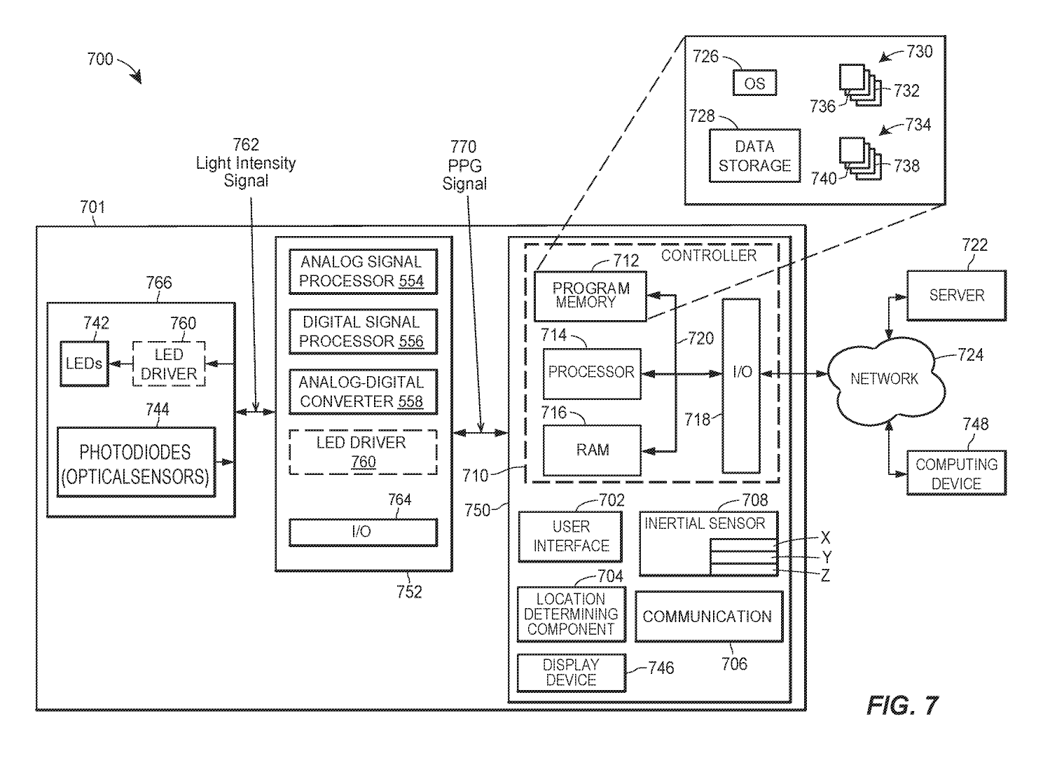

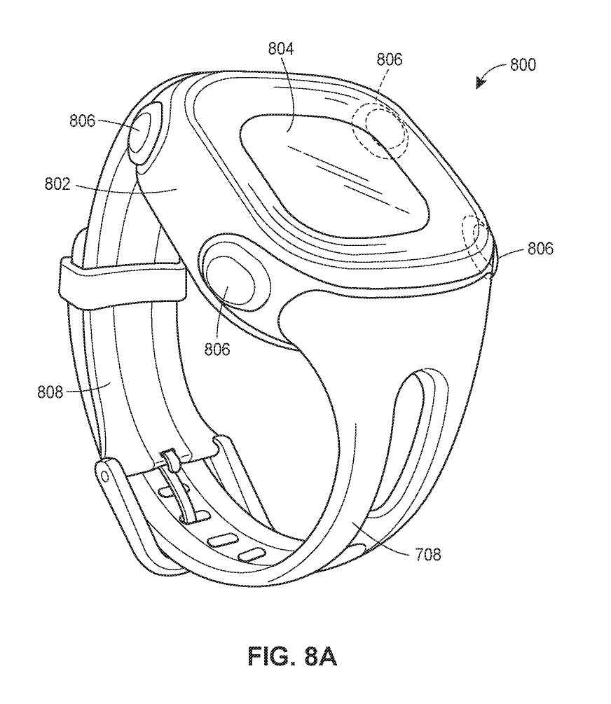

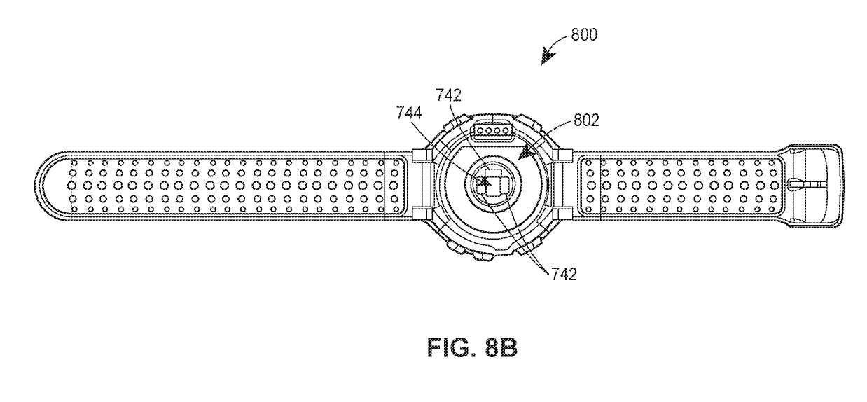

An example embodiment of a monitoring system 700 including a fitness monitor 701 capable of executing the methods and processes described above is illustrated in FIG. 7. The fitness monitor 701 includes an application processor 750 further including a user interface module 702, a location determining component 704 (e.g., a global positioning system (GPS) receiver, Assisted-GPS, etc.), a communication module 706, an inertial sensor 708 (e.g., accelerometer, gyroscope, etc.), and a controller 710. In embodiments, inertial sensor 708 may be external to application processor 750.

The fitness monitor 701 may be a general-use computer, wearable monitoring device (e.g., a wrist watch, ankle bracelet, etc.), a cellular phone, a smartphone, a tablet computer, or a mobile personal computer, capable of monitoring a physiological aspect of an individual as described herein. The fitness monitor 701 may be a thin-client device or terminal that sends processing functions to a server device 722 via a network 724. Communication via the network 724 may include any combination of wired and wireless technology. For example, network 724 may include a USB cable between fitness monitor 701 and computing device 748 to facilitate the bi-directional transfer of data between fitness monitor 701 and computing device 744.

The controller 710 of the application processor 750 may include a program memory 712, a microprocessor (MP) 714, a random-access memory (RAM) 716, and an input/output (I/O) circuitry 718, all of which may be communicatively interconnected via an address/data bus 720. Although the I/O circuitry 718 is depicted in FIG. 7 as a single block, the I/O circuitry 718 may include a number of different types of I/O circuits. The program memory 712 may include an operating system 726, a data storage device 728, a plurality of software applications 730, and/or a plurality of software routines 734. The operating system 726 of program memory 712 may include any of a plurality of mobile platforms, such as the iOS®, Android™, Palm® webOS, Windows® Mobile/Phone, BlackBerry® OS, or Symbian® OS mobile technology platforms, developed by Apple Inc., Google Inc., Palm Inc. (now Hewlett-Packard Company), Microsoft Corporation, Research in Motion (RIM), and Nokia, respectively.

The data storage device 728 of program memory 712 may include application data for the plurality of applications 730, routine data for the plurality of routines 734, and other data necessary to interact with the server 722 through the network 724. In particular, the data storage device 728 may include one or more determined reference values associated with the PPG signal 770 output in relation to physiological characteristic data and/or activities. The data storage device 730 may store information relating to locations of photodiodes 744 (optical sensors). For example, the data storage device 730 may include a lateral distance between two or more photodiodes 744 (optical sensors), which may be positioned on a rear surface of a housing of fitness monitor 701. Alternatively, the data storage device 730 may include locations of photodiodes 744 (optical sensors) that a processor 714 may utilize to determine a lateral distance between two or more photodiodes 744 (optical sensors), which may be positioned on a rear surface of a housing of fitness monitor 701.

Additional data stored within the data storage device 728 may include cardiac component data associated with the user and/or one or more other individuals. The cardiac component data may include one or more compilations of recorded physiological aspects of the user, including, but not limited to, a heartbeat, heart rate, heart-rate variability, speed, distance traveled, calculating calories burned, body temperature, and the like. In some embodiments, the controller 710 may also include, or otherwise be operatively coupled for communication with other data storage mechanisms (e.g., one or more hard disk drives, optical storage drives, solid state storage devices, etc.) that may reside within the fitness monitor 701 and/or operatively coupled to the network 524 and/or server device 722.

The fitness monitor 701 further includes a photometric front end circuit 752 communicably coupled between the application processor 750 and an optical sensing module 766. The optical sensing module 766 includes one or more LED(s) 742 and two or more photodiodes 744, and is configured for placement against or adjacent to the skin of a user when the fitness device 701 is attached to the user. In embodiments, the photometric front end 752 may include an analog signal processor 754, a digital signal processor 756, an analog-to-digital converter 558, a time slot controller 762, I/O circuitry 762, and an LED driver 760. The LED driver 560 may alternately be integrated within the optical sensing module 766 as indicated by the dotted lines of the component in FIG. 7. Further still, the LEDs 542, LED driver 760, and the photodiodes 744 of optical sensing module 766 may be integrated within the photometric front end 752.

Operation of the photometric front end 752 facilitates stimulating the one or more LED(s) 742 to emit light and measuring (sensing) any reflection of the emitted light returning from the user's skin received by two or more photodiodes 744, each of which generate and output an analog light intensity signal 762. The measured light intensity signals 762 may be analyzed by the analog signal processor 754 and/or digitized by the digital signal processor 756 and/or analog-to-digital converter 758 of the photometric front end 752. Control and/or data communication amongst the application processor 750, photometric front end 752, and optical sensing module is facilitated via the I/O circuitry 764. Photometric front end 752 may output to application processor 750 and/or store in data storage device 728 two or more PPG signals 770 in digital or digitized form corresponding to the analog light intensity signals 762 generated by photodiodes 744.

The one or more LEDs (e.g., emitters) 742 output visible and/or non-visible light, and the two or more photodiodes (optical sensors) 744 each receive reflections of the visible and/or non-visible light and generate the analog light intensity signal 762 based on the intensity of the received electromagnetic radiation of the reflected light. In embodiments, the one or more LEDs 742 may include any combination of green LEDs, red LEDs, and/or infrared LEDs that emit light into the user's skin located against or adjacent to the one or more LEDs 742. The two or more photodiodes 744 receive reflections of visible-light and/or infrared (IR) light emitted by the LEDs 742 into the user's skin and generate the light intensity signal 762 based on the received reflection of the emitted light from the user's skin located against or adjacent to the two or more photodiodes 744. The light intensity signals 762 generated by the two or more photodiodes 744 may be in analog form and communicated to the photometric front end 752 for signal processing and digitization. The photometric front end 752 may include filters for the light intensity signals 762 generated by the and analog-to-digital converters to digitize the light intensity signals 762 into digital PPG signals 770, which include a cardiac cycle signal component associated with the user's heartbeat.

Typically, the one or more LEDs 742 are positioned against or adjacent to the user's skin and emit light into the user's skin. The two or more photodiodes 744 are positioned near the one or more LEDs 742 to receive reflections of light emitted by the two or more LEDs 742 after reflection from the user's skin. The photometric front end 752 may output two or more digital PPG signals 770 based on corresponding analog light intensity signals 762 generated and output by each photodiode 744 based on an intensity of light reflected from the user's skin after being emitted by LEDs 742.

The intensity of measured light may be modulated by the cardiac cycle due to variation in tissue blood perfusion at a location against or adjacent to photodiodes 744 during the cardiac cycle. In user activity environments, the intensity of light reflections received (sensed) by photodiodes 744 may be impacted by a variety of factors, including, but not limited to, the inherent “noise” characteristic static and/or variable ambient light intensity, body motion at the measurement location, static and/or variable sensor pressure on the skin, motion of the fitness monitor 701 relative to the body at the measurement location, breathing, and/or light barriers (including hair, opaque skin layers, sweat, etc.). Relative to these sources, the cardiac cycle component of a PPG signal 770 may be very weak, frequently by one or more orders of magnitude.

The location determining component 704 may be a GPS receiver that is configured to provide geographic location information of the fitness monitor 701. The location determining component may be, for example, a GPS receiver such as those provided in various products by GARMIN®. Generally, GPS is a satellite-based radio navigation system capable of determining continuous position, velocity, time, and direction information. Multiple users may simultaneously utilize GPS. GPS incorporates a plurality of GPS satellites that orbit the earth. Based on these orbits, GPS satellites can relay their location to a GPS receiver. For example, upon receiving a GPS signal, e.g., a radio signal, from a GPS satellite, the fitness monitor 701 disclosed herein can determine a location of that satellite. The fitness monitor 701 can continue scanning for GPS signals until it has acquired a number, e.g., at least three, of different GPS satellite signals. The fitness monitor 701 may employ geometrical triangulation, e.g., where the watch utilizes the known GPS satellite positions to determine a position of the fitness monitor 701 relative to the GPS satellites. Geographic location information and/or velocity information can be updated, e.g., in real time on a continuous basis, for the fitness monitor 701.

In embodiments, inertial sensor 708 may incorporate one or more accelerometers positioned to determine an acceleration and direction of movements of fitness monitor 701. The accelerometer may determine magnitudes of acceleration in an X-axis, a Y-axis, and a Z-axis to measure the acceleration and direction of movement of fitness monitor 701 in each respective direction (or plane). It will be appreciated by those of ordinary skill in the art that a three dimensional vector describing a movement of the fitness monitor 701 through three dimensional space can be established by combining the outputs of the X-axis, Y-axis, and Z-axis accelerometers using known methods. Single and multiple axis models of the inertial sensor 508 are capable of detecting magnitude and direction of acceleration as a vector quantity, and may be used to sense orientation and/or coordinate acceleration of the user.

The photodiodes 744, location determining component 704 and the inertial sensors 508 may be referred to collectively as the “sensors” of the fitness monitor 701. It is also to be appreciated that additional location determining components 704 and/or inertial sensor(s) 708 may be operatively coupled to the fitness monitor 701. In embodiments, the fitness monitor 701 may also include or be coupled to a microphone incorporated with the user interface module 702 and used to receive voice inputs from the user while the fitness monitor 701 determines and monitors physiological characteristics and information of the user.

The communication module 706 may communicate with computing device 744 and/or server device 722 via any suitable wired or wireless communication protocol independently or using I/O circuitry 718. The wired or wireless network 724 may include a wireless telephony network (e.g., GSM, CDMA, LTE, etc.), one or more standard of the Institute of Electrical and Electronics Engineers (IEEE), such as 802.11 or 802.16 (Wi-Max) standards, Wi-Fi standards promulgated by the Wi-Fi Alliance, Bluetooth standards promulgated by the Bluetooth Special Interest Group, a near field communication standard (e.g., ISO/IEC 18092, standards provided by the NFC Forum, etc.), and so on. Wired communications are also contemplated such as through universal serial bus (USB), Ethernet, serial connections, and so forth.

The communication module 706 generally allows the user to upload data to, download data from, or adjust the settings of the fitness monitor 701. The communication module 706 may be wired or wireless and may include antennas, signal or data receiving circuits, and signal or data transmitting circuits. The communication module 706 may transmit and receive radio frequency (RF) signals and/or data and may operate utilizing communication standards such as ANT, ANT+, Bluetooth™ low energy (BLE), Near Field Communications (NFC), or the like. In various embodiments, the communication module 706 may transmit and receive data using the industrial, scientific, and medical (ISM) band at 2.4 gigahertz (GHz). Furthermore, in some embodiments, the communication module 706 may communicate with a wireless dongle that connects to the USB port of a desktop, laptop, notebook, or tablet computer, or other electronic device. An exemplary communication module 706 includes an nRF51922 RF integrated circuit (IC) from Nordic Semiconductor of Trondheim, Norway.

The fitness monitor 701 may be configured to communicate via one or more networks 724 with a cellular provider and an internet provider to receive mobile phone service and various content, respectively. Content may represent a variety of different content, examples of which include, but are not limited to: map data, which may include route information; web pages; services; music; photographs; video; email service; instant messaging; device drivers; real-time and/or historical weather data; instruction updates; and so forth.

The user interface 702 of the fitness monitor 701 may include a “soft” keyboard that is presented on a touchscreen display device 746 of the fitness monitor 701, an external hardware keyboard communicating via a wired or a wireless connection (e.g., a Bluetooth keyboard), and/or an external mouse, or any other suitable user-input device or component. As described earlier, the user interface 702 may also include or communicate with a microphone capable of receiving voice input from a vehicle operator as well as a display device 746 having a touch input.

With reference to the controller 710, it should be appreciated that controller 510 may include multiple microprocessors 714, multiple RAMs 716, and multiple program memories 712. The controller 710 may implement the RAM 716 and the program memories 712 as semiconductor memories, magnetically readable memories, and/or optically readable memories, for example. The one or more processors 714 may be adapted and configured to execute any of the plurality of software applications 730 and/or any of the plurality of software routines 734 residing in the program memory 712, in addition to other software applications. One of the plurality of applications 730 may be a client application 732 that may be implemented as a series of machine-readable instructions for performing the various functions associated with implementing the performance monitoring system as well as receiving information at, displaying information on, and transmitting information from the fitness monitor 701. The client application 732 may function to implement a system wherein the front-end components communicate and cooperate with back-end components as described above. The client application 732 may include machine-readable instructions for implementing the user interface 702 to allow a user to input commands to, and receive information from, the fitness monitor 701. One of the plurality of applications 730 may be a native web browser 736, such as Apple's Safari®, Google Android™ mobile web browser, Microsoft Internet Explorer® for Mobile, Opera Mobile™, that may be implemented as a series of machine-readable instructions for receiving, interpreting, and displaying web page information from the server device 722 or other back-end components while also receiving inputs from the fitness monitor 701. Another application of the plurality of applications 730 may include an embedded web browser 732 that may be implemented as a series of machine-readable instructions for receiving, interpreting, and displaying web page information from the server device 722 or other back-end components within the client application 732.

The client applications 730 or routines 734 may include one or more processes for determining the reference value(s) associated with the non-ideal operating characteristics of the fitness monitor 701. Namely, the analysis and determination of the reference value based on the emitter output values of the optical sensing module 766 in response to zero light impulse during the idle state of the fitness monitor. The client applications 730 or routines 734 may further include an accelerometer routine 738 that determines the acceleration and direction of movements of the fitness monitor 701, which correlate to the acceleration, direction, and movement of the user. The accelerometer routine 738 may receive and process data from the inertial sensor 708 to determine one or more vectors describing the motion of the user for use with the client application 732. In some embodiments where the inertial sensor 708 includes an accelerometer having X-axis, Y-axis, and Z-axis accelerometers, the accelerometer routine 738 may combine the data from each accelerometer to establish the vectors describing the motion of the user through three dimensional space. In some embodiments, the accelerometer routine 738 may use data pertaining to less than three axes. The client applications 730 or routines 734 may further include a velocity routine 740 that coordinates with the location determining component 704 to determine or obtain velocity and direction information for use with one or more of the plurality of applications, such as the client application 732, or for use with other routines. The user may also launch or instantiate any other suitable user interface application (e.g., the native web browser 736, or any other one of the plurality of software applications 730) to access the server device 722 to implement the monitoring process. Additionally, the user may launch the client application 732 from the fitness monitor 701 to access the server device 722 to implement the monitoring process.

After data has been gathered or determined by the sensors of the fitness monitor 701, the previously determined reference value may be utilized to adjust the PPG signals 770 utilized to determine the one or more physiological characteristics being monitored by the fitness monitor 701. The fitness monitor 701 may also implement time, frequency, pre- and post-conditioning, and time-variant filtering techniques. Once the extent of the adjustment has been assessed, a processed PPG signal can be determined and utilized by the processor to determine a physiological characteristic, such as blood pressure, or stress. The fitness monitor 701 may also transmit information associated with the cardiac component of the user. For example, the transmitted information may be sent to a remote server 722 capable of analyzing the data.

In embodiments where the fitness monitor 701 is a thin-client device, the server device 722 may perform one or more processing functions remotely that may otherwise be performed by the fitness monitor 701. In such embodiments, the server device 722 may include a number of software applications capable of receiving user information gathered by the sensors to be used in determining the a physiological characteristic, such as blood pressure, of the user. For example, processor 714 of the fitness monitor 701 may collect PPG signals from two or more photodiodes 744 and/or photometric front end 752 and gather information from data storage 728 as described herein, but instead of using the information locally, the fitness monitor 701 may send the information to the server device 722 for remote processing. The server device 722 may perform the analysis of the information to determine a physiological characteristic, such as blood pressure, of the user as described herein.

The disclosed techniques may be implemented in a wearable device, such as a watch, a mobile phone, a hand-held portable computer, a tablet computer, a personal digital assistant, a multimedia device, a media player, a game device, any combination thereof. For example, the wearable device may include a housing including a processor configured for use during fitness and/or sporting activities. FIGS. 8A and 8B illustrate views of one example embodiment of the wearable monitoring device of the monitoring system in accordance with one or more aspects described herein. The wearable device may be configured in a variety of ways to determine and provide fitness information, including one or more physiological characteristics of the user, such as blood pressure, as well as fitness functionality (e.g., steps taken, miles ran, etc.). The wearable monitoring device 800 includes a housing 802 configured to house, e.g., substantially enclose, various components of the monitor system. The housing 802 may be formed from a lightweight and impact-resistant material such as plastic, nylon, metal, or combinations thereof, for example. Portions of housing 802 may be formed from a non-conductive material, such a non-metal material. The housing 802 may include one or more gaskets, e.g., a seal, to make it substantially waterproof or water resistant. The housing 802 may include a location for a battery and/or another power source for powering one or more components of the wearable monitoring device 800. The housing 802 may be a singular piece or may include a plurality of sections. In some embodiments, the housing 802 may be formed from a conductive material, such as metal, or a semi-conductive material.

The wearable monitoring device 800 also includes an optical sensing module 810, as shown in FIG. 8B, including one or more light emitters (e.g., LEDs) of visible and/or non-visible light and one or more optical sensors (photodiodes) positioned to receive visible and/or non-visible light and generate an analog light intensity signal 762 (PPG signal 770) based on the intensity of the received light. A rear surface of housing 802 may include one or more LEDs and two or more optical sensors, which are positioned a lateral distance apart, such that a reflection of light emitted by the one or more LEDs may be received (sensed) by the two or more optical sensors positioned against or adjacent to the user's skin at the wrist where wearable monitoring device 800 is worn.

The wearable monitoring device 800 includes a display device 804. The display device 804 may include a liquid crystal display (LCD), a thin film transistor (TFT), a light-emitting diode (LED), a light-emitting polymer (LEP), and/or a polymer light-emitting diode (PLED). The display device 804 may be capable of displaying text and/or graphical information. The display device 804 may be backlit such that it may be viewed in the dark or other low-light environments. One example embodiment of the display device 804 is a 100 pixel by 64 pixel film compensated super-twisted nematic display (FSTN) including a bright white light-emitting diode (LED) backlight. The display device 804 may include a transparent lens that covers and/or protects components of the wearable monitoring device 800. The display device 804 may be provided with a touch screen to receive input (e.g., data, commands, etc.) from a user. For example, a user may operate the wearable monitoring device 800 by touching the touch screen and/or by performing gestures on the screen. In some embodiments, the touch screen may be a capacitive touch screen, a resistive touch screen, an infrared touch screen, combinations thereof, and the like. The wearable monitoring device 800 may further include one or more input/output (I/O) devices (e.g., a keypad, buttons, a wireless input device, a thumbwheel input device, a trackstick input device, and so on). The I/O devices may include one or more audio I/O devices, such as a microphone, speakers, and so on.

In accordance with one or more embodiments of the present disclosure, the wearable monitoring device 800 includes a user interface that generally allows the user to select which information is presented on the display and may include one or more control pushbuttons 806, or touch areas, such as a touchscreen. For example, the user may activate the user interface by pushing a button to cycle through a plurality of screens of data, wherein each screen of data may include informational items, such as those listed above. The user interface may be located either on the housing, on the display, or on the wrist band. As illustrated in FIG. 8A, four control buttons 806 are associated with, e.g., adjacent, the housing 802. While FIG. 8A illustrates four control buttons 806 associated with the housing 802, it is to be understood that the wearable monitoring device 800 may include more or less control buttons 806. Each control button 806 is configured to generally control a function of the wearable monitoring device 800. Functions of the mobile electronic device 800 may be associated with a location determining component and/or a performance monitoring component.

Functions of the wearable monitoring device 800 may include, but are not limited to, displaying a current geographic location of the wearable monitoring device 800, mapping a location on the display 804, locating a desired location and displaying the desired location on the display 804, and presenting physiological characteristic information based on a user pulse wave (PTT and/or PWV) including, but not limited to, blood pressure, hydration status, cardiac cycle signal, heartbeat signal, heart-rate signal or variability of heart rate signal for the user. User input may be provided from movement of the housing 802, for example, an inertial sensor(s), e.g., accelerometer, may be used to identify vertical, horizontal, and/or angular movement of the housing 802. In addition to or alternatively, user input may be provided from touch inputs identified using various touch sensing technologies, such as resistive touch or capacitive touch interfaces.

The wearable monitoring device 800 includes a strap 808 that enables one or more LEDs and one or more photodiodes to be securely placed against the skin of a user. The strap 808 is associated with, e.g., coupled to and/or integrated with, the housing 802 and may be removably secured to the housing 802 via attachment of securing elements to corresponding connecting elements. Some examples of securing elements and/or connecting elements include, but are not limited to, hooks, latches, clamps, snaps, and the like. The strap 808 may be made of a lightweight and resilient thermoplastic elastomer and/or a fabric, for example, such that the strap 808 may encircle a portion of a user without discomfort while securing the fitness monitor to the user. The strap 808 may be configured to attach to various portions of a user, such as a user's leg, waist, wrist, forearm, and/or upper arm.

In some embodiments, one or more optical sensors (photodiodes) of the monitoring system may be included in the strap and/or one or more optical sensors (photodiode) may be included in the housing of the wearable monitoring device, such that some optical sensors (photodiodes) may be included in the strap and/or remote to the housing. Placement of one or both optical sensors within the strap so as to be located against or adjacent to the user's wrist (the housing surface opposite the top surface typically including a display device) may provide a position for adequate detection of the pulse wave at two locations separated by a distance known to the processor. Regardless of the extremity chosen to attach wearable monitoring device 800 to the user, the processor may determine one or more physiological characteristics of the user (e.g., blood pressure, stress, etc.) based on PTT and/or PWV, at least partially based on known positions of or lateral distance between two or more photodiodes positioned against or adjacent to the user's skin at a location to enable detection of a pulse wave.

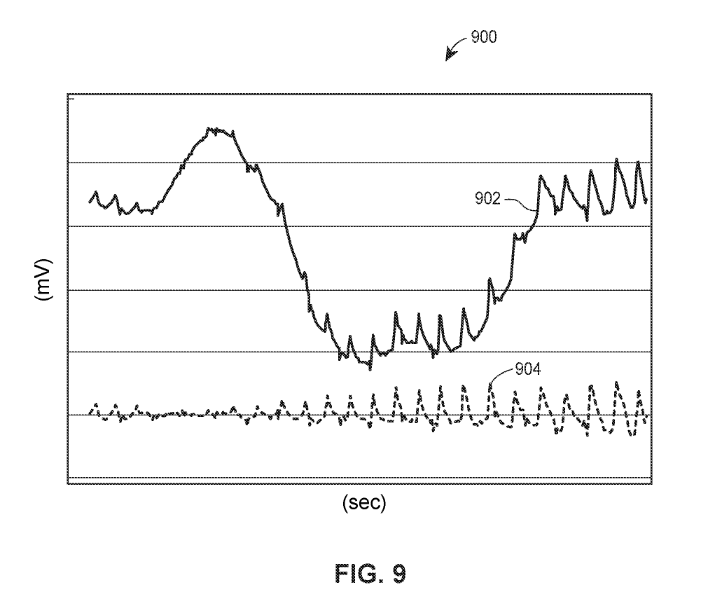

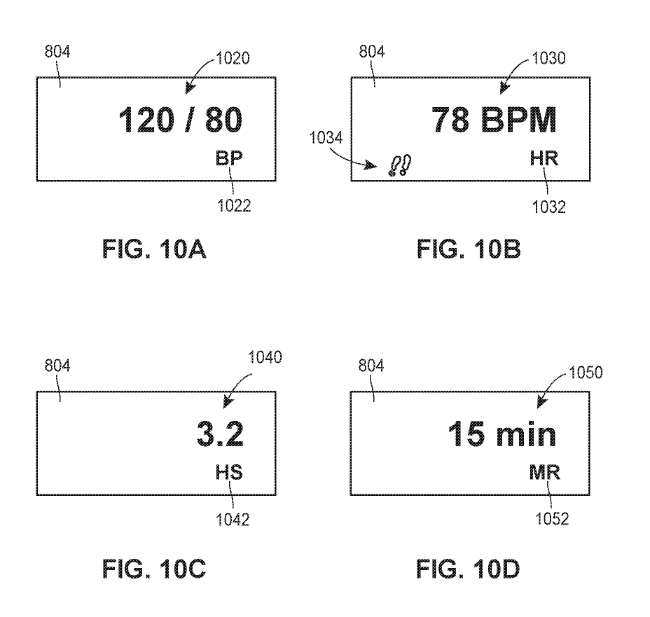

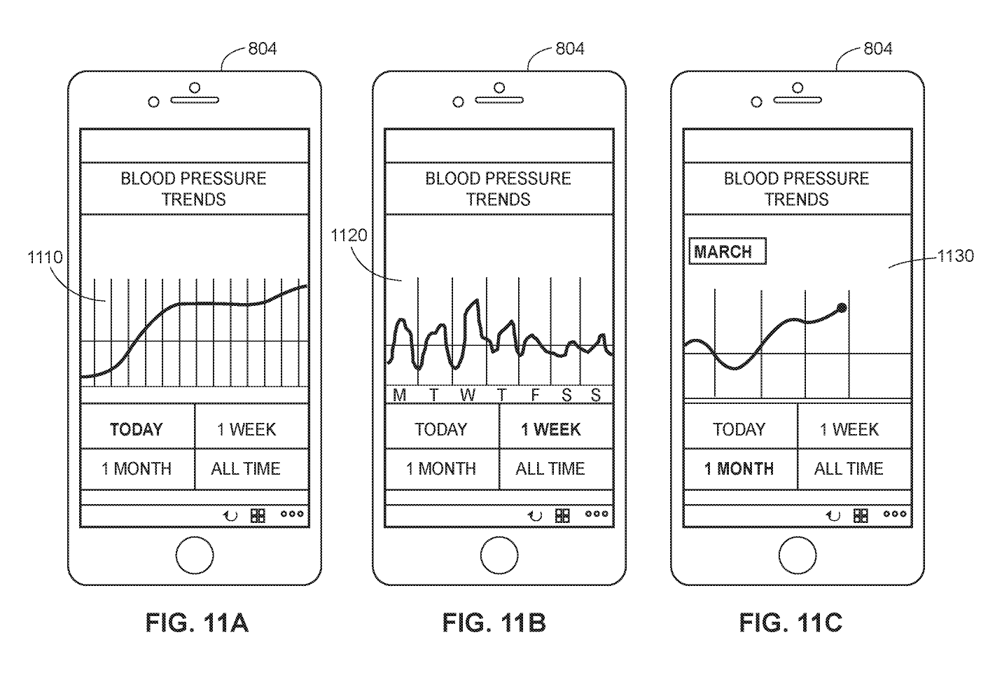

In embodiments, the processor may implement techniques to remove motion artifacts present in a PPG signal. Because the two or more optical sensors may be rigidly attached to each other (e.g., integrated within a housing of the wearable monitoring device), it is expected that the optical sensors would both experience similar, if not the same, motion artifact. Because a motion artifact may be sensed by both optical sensors at (slightly) different times, the processor may subtract one PPG signal from the other PPG signal to remove the motion artifact. FIG. 9 illustrates a plot 900 including a differential signal 902, similar to differential signal 606 of FIG. 6B, having distinct peaks for each heartbeat and a filtered signal 904, which cancels the slow DC offset seen as the slow rise and fall in the differential signal 902. The differential signal 902 is the result of the subtraction of a first PPG signal provided by a first optical sensor (located closer to a user's heart) from a second PPG signal provided by second optical sensor (located farther from the user's heart in relation to the first optical sensor). The filtered signal 904 illustrates the filtered differential signal 902 that has been passed through a “high-pass” filter. The high-pass filter may be used to remove a user's breathing. Thus, the processor may calculate a mathematical difference between the signals from each optical sensor and the primary signal that remains should have a detectable pulse signal under noisy signal conditions.

The display device 804 may be contained by the housing of the wearable monitoring device or remote from the wearable monitoring device and included within a mobile wireless communication device (wirelessly coupled with the processor using a transceiver). The display device 804, as seen in FIGS. 9A-9D, generally presents information associated with the user's physiological characteristic and/or activity. For example, the display device 804 in FIG. 10A may present the user's blood pressure 1020 (systolic/diastolic), as indicated by use of indicator “BP” 1022. In FIG. 10B, the display device 804 displays the user's activity (e.g., skating, skiing, rowing, cycling, walking, jogging, running, aerobics, or any other physical activity), as indicated by use of activity indicator 1034, and heart-rate 1030 (beats/minute) as indicated by indicator “HR” 1032. In FIG. 10C, the display device 804 displays the user's hydrations status 1040, as indicated by use of indicator “HS” 1042. In FIG. 10D, the display device 804 displays the user's monitoring rate 1050 (period, frequency), as indicated by use of indicator “MR” 1052.

Additionally, the processor may control the presentation of the physiological characteristic information in an intuitive manner. For example, the processor may determine (and store in a memory) a simple average of a user's systolic blood pressure and diastolic pressure or a mean arterial pressure (MAP) and control a display device (within a housing of a wearable monitoring device or a remote smartphone) to present this information in one or more periods of time to help a user track the determined blood pressure trend over time. Some examples of physiological characteristic information of the user displayed remote from the wearable monitoring device are illustrated in FIGS. 11A (daily blood pressure measurements), 11B (weekly blood pressure measurements), and 11C (monthly blood pressure measurements). FIG. 11A depicts a display device 804 controlled by a processor to present user interface 1110 including data associated with a physiological characteristic over a single day. FIG. 11B depicts a display device 804 controlled by a processor to present user interface 1120 including data associated with a physiological characteristic over one week. FIG. 11C depicts a display device 804 controlled by a processor to present user interface 1130 including data associated with a physiological characteristic over one month.

It is to be understood that there are ultimately many uses for continuous blood pressure measurement on a wrist-worn wearable monitoring device. For example, pre-hypertension, hypertension, hydration status, stress level, fitness metrics, and various wellness/medical conditions that present high or low blood pressure may be identified by wearable monitoring system described herein. For instance, the processor may use the determined PWV (and/or PTT) and a determined heart rate (by counting the number of heart beats sensed within a period of time) to determine a user's hydration status.

Additionally, the secure and comfortable attachment of the wearable monitoring system to the user ensures accurate measurements during high levels of physical activity. Further, the nondescript measuring aspects of the optical sensors provides measurement periods that are essentially undetectable by the user, which will be less distractive to the user during periods of sleep and/or when high levels of concentration may be required, e.g., while working.

The applications and benefits of the systems, methods, and techniques described herein are not limited to only the above examples. Many other applications and benefits are possible by using the systems, methods, and techniques described herein. Thus, many modifications and variations may be made in the techniques and structures described and illustrated herein without departing from the spirit and scope of the present invention. Accordingly, it should be understood that the methods and apparatus described herein are illustrative only and are not limiting upon the scope of the invention.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Also, unless a claim element is defined by reciting the word “means” and a function without the recital of any structure, it is not intended that the scope of any claim element be interpreted based on the application of 35 U.S.C. §112(f) and/or pre-AIA 35 U.S.C. §112, sixth paragraph.

Moreover, although the foregoing text sets forth a detailed description of numerous different embodiments, it should be understood that the scope of the patent is defined by the words of the claims set forth at the end of this patent. The detailed description is to be construed as exemplary only and does not describe every possible embodiment because describing every possible embodiment would be impractical, if not impossible. Numerous alternative embodiments could be implemented, using either current technology or technology developed after the filing date of this patent, which would still fall within the scope of the claims.

What is claimed:

1. A wearable monitoring device capable of being attached to the extremity of a user and determining a physiological characteristic of the user, the device comprising:
a housing containing a first photodiode and a second photodiode, at least one light-emitting diode (LED), and a processor;
the at least one LED configured to output light into the user's extremity;
the first photodiode positioned proximate to the at least one LED, the first photodiode configured to detect a pulse wave of the user based on a reflection of the light from the user's extremity and generate a first photoplethysmograph (PPG) signal;
the second photodiode proximate to the at least one LED and spaced a lateral distance from the first photodiode within the housing, the second photodiode configured to detect the pulse wave of the user based on a reflection of the light from the user's extremity and generate a second PPG signal; and
the processor coupled to the first and second photodiodes, the processor configured to:
receive the first and second PPG signals,
determine a pulse-transit-time of the user based on the generated first and second PPG signals, and
calculate the physiological characteristic of the user based on the calculated pulse-transit-time and the lateral distance between the first and second photodiodes; and
wherein the second photodiode detects the pulse wave after the first photodiode when the device is attached to the extremity of the user.

2. The wearable monitoring device of claim 1, wherein the physiological characteristic is a systolic pressure, diastolic pressure, mean arterial pressure, and/or hydration status of the user.

3. The wearable monitoring device of claim 1, further comprising a display contained by the housing, the display coupled to the processor and capable of displaying the calculated physiological characteristic of the user.

4. The wearable monitoring device of claim 3, further comprising a remote display remote from the housing, the remote display coupled to the processor and capable of displaying the calculated physiological characteristic of the user.

5. The wearable monitoring device of claim 3, further comprising:
an accelerometer coupled to the processor and configured to detect user movement, wherein a display format of the calculated physiological characteristic is based on the detected user movement.

6. The wearable monitoring device of claim 1, further comprising:
an accelerometer coupled to the processor and configured to detect user movement, wherein an operating mode for calculating the physiological characteristic of the user is based on the detected user movement.

7. The wearable monitoring device of claim 1, further comprising a fastener capable of attaching the wearable mobile device to the extremity of the user.

8. The wearable monitoring device of claim 1, wherein lateral distance between the first and second photodiodes is 5 to 25 millimeters.

9. The wearable monitoring device of claim 2, further comprising:
data stored on a memory device coupled to the processor, the data including a correlation between systolic pressure, diastolic pressure, and/or mean arterial pressure with pulse-transit-time and/or pulse wave velocity derived from pulse-transit-time.

10. The wearable monitoring device of claim 2, further comprising:
data stored on a memory device coupled to the processor, the data including a correlation between hydration status and pulse-transit-time and/or pulse wave velocity derived from the pulse-transit-time.

11. A wearable monitoring device capable of being attached to the extremity of a user and determining a physiological characteristic of the user, the device comprising:
a first photodiode spaced apart a lateral distance from a second photodiode when the device is attached to the extremity of the user, the lateral distance extends along the longitudinal axis of the extremity of the user between the first photodiode and the second photodiode;
at least one light-emitting diode (LED) proximate the first photodiode and the second photodiode and configured to output light received by the first and second photodiodes;
the first photodiode detecting a pulse wave of the user and generating a first photoplethysmograph (PPG) signal;
the second photodiode detecting the pulse wave of the user and generating a second PPG signal; and
a processor coupled to the first and second photodiodes, the processor configured to:
calculate the physiological characteristic of the user based on the generated first and second PPG signals and the lateral distance between the first and second photodiodes.

12. The wearable monitoring device of claim 11, wherein the physiological characteristic includes systolic pressure, diastolic pressure, mean arterial pressure, and/or hydration status of the user.

13. The wearable monitoring device of claim 11, further comprising a housing containing the processor and at least one of the first and second photodiodes.

14. The wearable monitoring device of claim 13, further comprising a display contained by the housing, the display coupled to the processor and capable of displaying the calculated physiological characteristic of the user.

15. The wearable monitoring device of claim 13, further comprising a remote display remote from the housing and capable of displaying the calculated physiological characteristic of the user.

16. The wearable monitoring device of claim 11, further comprising:
a housing containing the processor and at least one of the first and second photodiodes;
a display contained by the housing, the display coupled to the processor and capable of displaying the calculated physiological characteristic of the user; and
an accelerometer coupled to the processor, wherein a display format of the calculated physiological characteristic is based on user movement detected by the accelerometer.

17. The wearable monitoring device of claim 11, further comprising:
a housing containing the processor and at least one of the first and second photodiodes;
a display contained by the housing, the display coupled to the processor and capable of displaying the calculated physiological characteristic of the user; and
an accelerometer coupled to the processor, wherein an operating mode for calculating the physiological characteristic is based on user movement detected by the accelerometer.

18. A wearable monitoring device capable of being attached to the extremity of a user and determining blood pressure of the user, the device comprising:
a first photodiode spaced apart a lateral distance from a second photodiode when the device is attached to the extremity of the user, the lateral distance extends along the longitudinal axis of the extremity of the user between the first photodiode and the second photodiode;
at least one light-emitting diode (LED) proximate the first photodiode and the second photodiode and configured to output light received by the first and second photodiodes;
the first photodiode detecting a pulse wave of the user and generating a first signal;
the second photodiode detecting the pulse wave of the user and generating a second signal;
a processing means for calculating the blood pressure of the user based on the generated first and second signals and the lateral distance between the first and second photodiodes; and
a display coupled to the processing means for calculating the blood pressure of the user and displaying the calculated blood pressure.

19. The wearable monitoring device of claim 18, wherein the processing means for calculating the blood pressure of the user includes a processor coupled to the first and second photodiodes and configured to calculate a pulse-transit-time of the user based on the generated first and second signals.

20. The wearable monitoring device of claim 19, further comprising:
data stored on a memory device coupled to the processing means for calculating the blood pressure of the user, the data including a correlation between systolic pressure, diastolic pressure, and/or mean arterial pressure with pulse-transit-time and/or pulse wave velocity derived from pulse-transit-time.
